(19) United States
(12) Patent Application Publication
(10) Pub. No.: US 2017/0128412 A1
(43) Pub. Date: May 11, 2017

(54) SYSTEM AND METHOD FOR IMPROVED BLOOD CIRCULATION AND MAINTAINING MITOCHONDRIAL ENERGY PRODUCTION

(71) Applicants: Satoru Sugiyama, Nagoya (JP); Takado Ishii, Yokohama (JP); Takayuki Ozawa, Shimada (JP)

(72) Inventors: Satoru Sugiyama, Nagoya (JP); Takado Ishii, Yokohama (JP); Takayuki Ozawa, Shimada (JP)

(21) Appl. No.: 15/235,068

(22) Filed: Aug. 11, 2016

Related U.S. Application Data
(60) Provisional application No. 62/203,841, filed on Aug. 11, 2015.

(51) Int. Cl. A61K 31/355; A61K 47/18; A61K 47/26; A61K 9/00; A61K 9/08

(52) U.S. Cl. CPC A61K 31/355; A61K 9/0019; A61K 9/08; A61K 47/26; A61K 47/183

(57) ABSTRACT

The disclosure relates to compositions, methods and systems for improving blood circulation and/or for the treatment of acute poor circulation of the liver, kidneys, heart, brain, and other organs utilizing highly concentrated tocotrienols/tocotrienol derivatives. The compositions, methods and systems of the disclosure may also be applicable in the treatment of chronic tissue hypoxemia, organ damage as a result of cell death, dementia, ulceration of the skin, and other such internal organ/brain/skin failure.

### CROSS-REFERENCE TO RELATED APPLICATIONS

This application claims the benefit under 35 U.S.C. §119(e) of U.S. Provisional Application No. 62/203,841 filed Aug. 11, 2015 with a docket number of APT-0002.PO, which is hereby incorporated by reference herein in its entirety.

### STATEMENT REGARDING FEDERALLY SPONSORED RESEARCH OR DEVELOPMENT

Not Applicable.

### BACKGROUND

The disclosure relates to compositions, methods and systems for improving blood circulation and/or for the treatment of acute poor circulation of the liver, kidneys, heart, brain, and other organs utilizing highly concentrated tocotrienols/tocotrienol derivatives, and/or for maintaining mitochondrial energy production. The compositions, methods and systems of the disclosure may also be applicable in the treatment of chronic tissue hypoxemia, organ damage as a result of cell death, dementia, ulceration of the skin, and other such internal organ/brain/skin failure. The disclosure also relates to compositions, methods and systems for reversing deteriorated cellular energy metabolism (or deteriorated mitochondrial function) caused by poor circulation of the liver, kidneys, heart, brain, and other organs utilizing highly concentrated tocotrienols/tocotrienol derivatives and several amino acids.

What is needed are methods and systems that are efficient at improving blood circulation for the treatment of acute poor circulation of the liver, kidneys, heart, brain, and other organs. As will be seen, the disclosure provides such methods and systems that can treat acute poor circulation of the liver, kidneys, heart, brain, and other organs in an effective and elegant manner.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawing where:

FIG. 1 illustrates the general chemical structure of tocotrienols, specifically alpha(α)-tocotrienol, according to one implementation consistent with the teachings and principles of the disclosure.

### DETAILED DESCRIPTION

The disclosure extends to compositions, methods, and systems for improving blood circulation and/or the treatment of acute poor circulation of the liver, kidneys, heart, brain, and other organs. In the following description of the disclosure, reference is made to the accompanying drawing, which forms a part hereof, and in which is shown by way of illustration specific implementations in which the disclosure is may be practiced. It is understood that other implementations may be utilized and structural changes may be made without departing from the scope of the disclosure.

There are many unknowns when it comes to organ failure brought on by hypoxemia, respiratory failure, or poor blood circulation. There are currently no treatments that target the specific cause of organ failure due to poor circulation. The compositions, methods, and systems of the disclosure are proposed for use as a treatment for acute and chronic failure of the liver, kidneys, heart, brain, skin, and other organs. The compositions, methods, and systems of the disclosure are also proposed for use in preventing the progression of further tissue damage by increasing blood circulation, prolonging cell life, and maintaining mitochondrial energy production.

Vitamin E is widely recognized as an important lipid-based antioxidant that can protect unsaturated fatty acids in the cell membrane. Although a-tocopherol is generally recognized as the isoform with the highest vitamin E activity, tocotrienol has been shown to have much higher free radical scavenging ability i.e. antioxidant activity than a-tocopherol within the cell. This is due to the higher penetration of tocotrienol molecules into the cell while α-tocopherol mainly resides on the plasma membrane. As such, tocotrienol is capable of protecting important organelles like the mitochondria (responsible for energy production) and DNA (genetic code) from damage induced by oxidative stress better than a-tocopherol.

Increasingly, recent studies show that tocotrienol shows greater clinical benefits in cardiovascular, brain, liver, and women's health. Numerous studies indicate that cholesterol and triglyceride levels can be controlled with prolonged consumption of palm-derived tocotrienol with the effect mainly due to gamma and delta tocotrienol. These two tocotrienol isomers are also responsible for controlling inflammatory responses in the body especially in conditions like metabolic syndrome, pre-diabetes, obesity, fatty liver etc.

Alpha tocotrienol, which is abundant in palm, has been extensively researched for brain health benefits, including reduction of post-stroke injury and recovery. A recent study also found that patients with mild cognitive impairment and Alzheimer's disease have low levels of vitamin E in their blood plasma, in particular alpha and gamma tocotrienol.

The technology of the disclosure is an improvement to the art in that it utilizes a processed and highly concentrated tocotrienol to improve mitochondrial antioxidant function and blood circulation. Further, the technology of the disclosure combines the radical scavenging ability of tocotrienol with certain amino acids to improve blood circulation, mitochondrial energy production, and prolong cell life. Tocotrienol can work with certain amino acids to enhance the benefits of each in terms of improving mitochondrial energy production and prolonging cell life.

It has been found that when exposing human cell cultures to low-acidity and low-nutrient conditions, highly concentrated tocotrienol production increases. The citric acid cycle, the final stage of metabolism, contains dehydrogenase enzymes. The hydrogen cleaved off of a substrate by these dehydrogenases are combined with oxygen in the mitochondrial electron transport chain to produce water. This cellular respiration is the means by which cells harvest energy to survive. However, when oxygen intake is hindered by organ failure or electron transport disruption, the metabolism introduces tocotrienol, which is turned into fumaric acid, succinic acid, and then propionic acid. It is thought that cells are able to survive without oxygen by utilizing this cycle. The catalyst comprising tocotrienol and select amino acids increases the concentration of succinic and fumaric acid in the mitochondria, and this is why it is believed that tocotrienol and select amino acids may aid in the survival of cells that are unable to respire aerobically. That is, tocotrienol and select amino acids can aid in mitochondrial energy production and the prolonging of cell life when cells exist in a low-oxygen or hypoxic condition.

Further, when oxygen intake is hindered by organ failure or electron transport disruption, inadequate oxygen supply produces a large number of oxygen free radicals, which in turn results in further mitochondria damage leading to acute organ failure. Tocotrienol is a particularly effective scavenger of oxygen free radicals, and as such tocotrienol can be an effective component for prolonging cell life and maintaining mitochondrial energy production when the cell exists in a hypoxic condition. Further supplementation of amino acids can be metabolized as a mitochondrial citric acid cycle substrate, such as fumaric acid, succinic acid, and then propionic acid. These acids may then be used for energy production. It is thought that cells are able to survive under low oxygen concentration through utilizing this cycle.

The citric acid cycle—also known as the tricarboxylic acid (TCA) cycle or the Krebs cycle—is a series of chemical reactions used by all aerobic organisms to generate energy through the oxidation of acetyl-CoA derived from carbohydrates, fats and proteins into carbon dioxide and chemical energy in the form of adenosine triphosphate. In addition, the cycle provides precursors of certain amino acids as well as the reducing agent NADH that is used in numerous other biochemical reactions. The continued successful completion of citric acid cycle is imperative for maintaining cell life. It is a notable aspect of the disclosure that tocotrienol and select amino acids can work together to maintain mitochondrial energy production through the citric acid cycle.

The proposed improvement made by the disclosure is as follows: highly-concentrated tocotrienol or a tocotrienol derivative, amino acids and their physiologically tolerable salts, and highly-concentrated tocotrienol/tocotrienol derivative-containing compositions and medications used to treat acute and chronic organ failure or brain and skin degeneration (compositions and medications used to treat organ failure/brain and skin degeneration will henceforth be referred to as “organ failure drugs”). The proposed improvement is further an effective treatment for maintaining mitochondrial energy production, improving blood circulation, and prolonging cell life.

This new acute and chronic organ failure composition aids cells, which have undergone metabolic switch due to being thrown into a low-oxygen/low-nutrient state as a result of poor circulation or respiratory failure. Through preserving the inner-cellular production of energy, loss of organ function, cell death, and blood clot progression can be halted. This composition can be utilized, for example, as a treatment for diseases of the organs and skin. This acute and chronic organ failure composition is most effectively administered intravenously for acute cases, and as a tablet or powder and water solution for chronic cases.

It is best to include one, two, or more of the following amino acids in this composition: arginine, glutamine, glutamic acid, proline, serine, cysteine, histidine, tryptophan, tyrosine, and their physiologically tolerable salts. For pharmacological salts, inorganic hydrochloric and sulfuric acid salts and organic acetic and formic acid salts are options. Additionally, sodium, potassium, and other alkali metal salts could be proposed as well.

When combining two or more amino acids, it is best to consider the metabolic effects of the amino acids in order to increase mitochondrial fumaric and succinic acid in the target tissue. It is also important to consider the pH of the solvent, as a neutral final solution is ideal. Further, the pH of the one or more amino acids, along with the pH of the solvent will affect the final pH of the solution. As such, the ratio of the one or more amino acids may be optimized to maintain a final neutral composition. For example, if the solution comprises two amino acids, where one amino acid is slightly acidic and the other amino acid is slightly basic, then the amino acids may be present in equimolar concentrations to ensure a final neutral solution. That is, the acidity of the amino acids should be considered when determining the concentration of each of the amino acids, and the proportions of each amino acid may be adjusted such that the final composition is neutral.

The highly concentrated tocotrienol or tocotrienol derivatives included in this composition may counteract the symptoms of organ failure, poor cell respiration, poor mitochondrial energy production, premature cell death, etc. For example, the disclosed composition may be used for a patient with brain ischemia or for expansion of an ischemic penumbra after a stroke. Further, the composition may reduce cell death caused by, for example, angina pectoris, a myocardial infarction, bedsores caused by hindered blood flow, or other acute conditions. The composition may further be provided to a patient with heart, live, or kidney failure, dementia, or other chronic conditions.

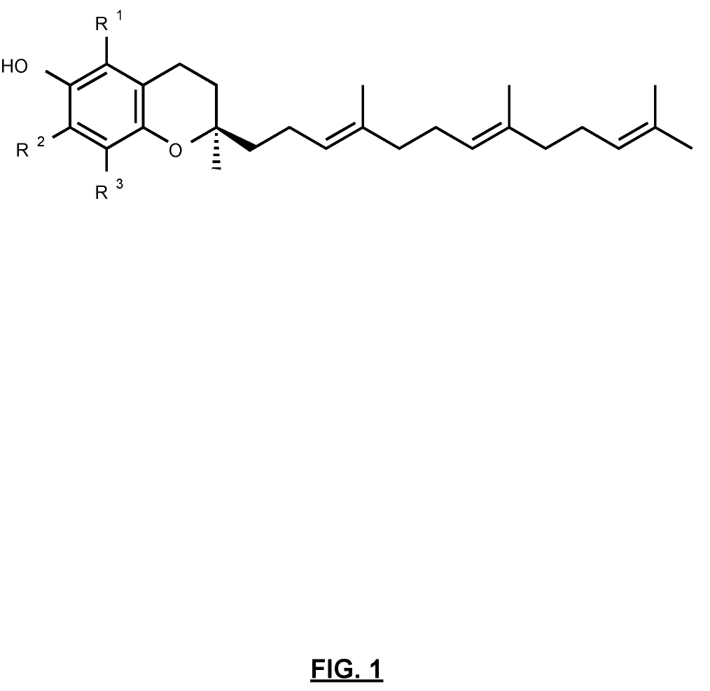

Tocotrienol and tocotrienol derivatives are represented in their general form in FIG. 1. FIG. 1 illustrates the general chemical structure of tocotrienols, specifically alpha(α)-tocotrienol.

It will be appreciated that tocotrienols are members of the vitamin E family. Vitamin E is an essential nutrient for the body. Vitamin E is made up of four tocopherols (alpha, beta, gamma, and delta) and four tocotrienols (alpha, beta, gamma, and delta). The slight difference between tocotrienols and tocopherols lies in the unsaturated side chain of tocotrienols, having three double bonds on the farnesyl isoprenoid tail.

Tocotrienols are compounds found in select vegetable oils, including rice bran oil and palm oil, wheat germ, barley, saw palmetto, anatto, and certain other types of seeds, nuts, grains, and the oils derived from them. However, tocotrienols are not found in highly concentrated forms in nature. This variant of vitamin E typically only occurs at very low levels in nature. To obtain highly concentrated forms of tocotrienols, significant processing must occur. Further, to obtain purified tocotrienols, particularly at highly concentrated levels, the natural products comprising low levels of tocotrienols must undergo very significant processing.

Chemically, vitamin E in all of its forms functions as an antioxidant, or scavenger of free radicals. All of the tocotrienol and tocopherol isomers have this antioxidant activity due to the ability to donate a hydrogen atom (a proton plus electron) from the hydroxyl group on the chromanol ring, to a free radical in the body. This process inactivates the free radical by effectively donating a single unpaired electron (which comes with the hydrogen atom) to the radical. Thus, one model for the function of vitamin E in the body is that it protects cell membranes, active enzyme sites, and DNA from free radical damage.

Tocotrienols have only a single chiral center, which exists at the 2′ chromanol ring carbon, at the point where the isoprenoid tail joins the ring. The other two corresponding centers in the phytyl tail of the corresponding tocopherols do not exist due to tocotrienol's unsaturation at these sites. Tocotrienols extracted from natural sources always consist of the dextrorotatory enantiomers only. These dextrorotatory stereoisomers are generally abbreviated as the “d-” forms, for example, “d-tocotrienol” or “d-alpha-tocotrienol”. In theory, the unnatural “l-tocotrienol” (levorotatory) forms of tocotrienols could exist as well, which would have a 2S (rather than 2R) configuration at the molecules' single chiral center.

Tocotrienols and tocotrienol derivatives have three units on the isoprene chain, and may have a higher cellular concentration than coenzymes having more units on the isoprene chain. That is, tocotrienols and tocotrienol derivatives may better penetrate the cell membrane and enter the intracellular fluid than comparable molecules. Tocotrienols may be more effective at penetrating the cell membrane than larger comparable molecules, such as for example Coenzyme Q10, which comprises ten units on the isoprene chain. Tocotrienols may also be more effective at penetrating the cell membrane and improving conditions in the cell than, for example, commonly used tocopherols.

Tocotrienol can be incorporated into cellular membranes where it can effectively inhibit the peroxidation of lipids. Tocotrienols can scavenge the chain-propagating peroxyl radicals and function as effective antioxidants. Nuclear magnetic resonance studies have indicated that tocotrienol is located closer to the membrane surface, which may facilitate recycling and lead to tocotrienol exhibiting particularly high antioxidant activity. Furthermore, tocotrienol has a stronger disordering effect on membranes than, for example, a-tocopherol, and is distributed more uniformly within the membrane. These properties likely enhance the interaction of tocotrienol with lipid radicals.

Studies indicate that tocotrienols exhibit an inhibition rate for linoleate hydroperoxide generation of about 40%, whereas Coenzyme Q10, for example, exhibits no such inhibition. Linoleate hydroperoxide is a cell membrane component, and protection of membrane linoleate peroxidation may lead to maintaining cell membrane activity. That is, the ability of tocotrienols to inhibit the peroxidation of linoleate hydroperoxide may lead to maintaining the cell membrane and thereby prolonging cell life.

Further, tocotrienol is an effective antioxidant because it can effectively scavenge free radicals. This attribute is particularly effective for preventing cell death when a cell exists in a hypoxic condition. Under normal conditions when a cell has an adequate oxygen supply, an adequate ratio of hydrogen peroxide is maintained such that oxygen radical formation is limited. However, in low-oxygen conditions, such as ischemia, there is an induced inadequate ratio of hydrogen peroxide, and the formation of oxygen radicals is increased. Oxygen radicals can be highly damaging to the cell and the organism as a whole, and it is imperative to cell life that the concentration of free radicals is minimized. Accordingly, the need for a free radical scavenger, such as tocotrienol, is increased in a hypoxic condition. Tocotrienol, a compound that can pass the cell membrane, can be an effective scavenger of free radicals in the mitochondria and throughout the cell.

Further, in hypoxic conditions, the supply of hydrogen derived from tricarboxylic acid is restricted. Exogenously administered tricarboxylic acid does not enter the mitochondria, but certain amino acids can easily enter the mitochondria and provide protective effects during mitochondria energy production in a hypoxic condition. For example, aspartic acid in particular can be converted to oxaloacetate, which is an intermediate of the citric acid cycle. Further for example, arginine acid and glutamic acid can be converted to oxoglutarate, which is another intermediate of the citric acid cycle. These amino acids can easily pass the cell membrane and enter the mitochondria, and then convert into the aforementioned organic acids that function as intermediaries in the continuation of the citric acid cycle. That is, glutamic acid, arginine acid, and aspartic acid may be particularly effective for the continuation of the citric acid cycle during a hypoxic condition. As stated above, the continued successful completion of the citric acid cycle is imperative for energy production, prolonging cell life, prolonging organ life, and thereby prolonging the life of the organism.

The combination of tocotrienol and certain amino acids can be particularly effective at preventing cell death during a hypoxic condition. That is, tocotrienol may function as an effective scavenger of free radicals when the ratio of oxygen free radicals is elevated during a hypoxic condition. The maintenance of free radical levels can be important for maintaining cell life. Further, certain amino acids, for example glutamic acid, arginine acid, and aspartic acid, can pass the cell membrane and enter the mitochondria to convert into intermediates of the citric acid cycle, and thereby enable the citric acid cycle to continue during a hypoxic condition. That is, particularly during a hypoxic condition, tocotrienol and certain amino acids may work together to effectively maintain cell life.

Tocotrienol can work cohesively with certain amino acids to prolong cell life, particularly in low-oxygen conditions. In low-oxygen conditions, there is an increased level of oxygen free radicals, as discussed above. Tocotrienol can effectively scavenge the free radicals and prevent damage, even cell death, caused by high levels of free radicals. Further, certain amino acids can enter the mitochondria and allow the citric acid cycle to continue by converting into intermediates of the citric acid cycle. Cell processes are dependent on the energy produced by the citric acid cycle, and thus cell life is prolonged if the citric acid cycle can continue even under difficult hypoxic conditions. Therefore, tocotrienol and certain amino acids together can prolong cell life and decrease the likelihood of cell death. That is, neither of tocotrienol or certain amino acids alone are as effective as they are together at prolonging cell life under hypoxic conditions.

In one embodiment, the composition comprises tocotrienols or tocotrienol derivatives in combination with glutamine, arginine, and aspartic acid. This combination of tocotrienols and amino acids may be particularly compatible with the citric acid cycle that occurs in the mitochondria in eukaryotic cells.

Pharmacologically tolerable salts include hydrochloric, sulfuric, and other inorganic acid salts, organic salts such as those of acetic and fumaric acid, sodium, potassium, and other alkali metal salts.

In one embodiment, the composition may be provided for the treatment of acute organ failure. When the composition is provided for the quick treatment of acute organ failure, it is most efficiently administered intravenously. The composition may be dissolved in a physiological saline solution or 5% sugar solution, or any other suitable solution, when it is prepared for intravenous administration. Further, the composition may be prepared immediately before administration to the patient.

In a further embodiment, the composition may be provided for the treatment of chronic organ failure, poor mitochondrial energy production, poor blood circulation, or chronic premature cell death. When the composition is provided for such conditions, it may be conveniently administered in a tablet form or a powder and aqueous solution form of the composition.

In one embodiment, the composition is provided for intravenous or intramuscular administration. In such an embodiment, the composition may comprise from about 0.2 g to about 4 g of amino acid per 100 mL of solution. The composition may further comprise from about 0.5 mg to about 2 mg of highly concentrated tocotrienols or tocotrienol derivatives per 200 mL to about 500 mL of solution. In one embodiment, the composition is provided for intravenous or intramuscular administration from about two to about four times per day.

In one embodiment, the composition is provided in a tablet form or a powder plus aqueous solution form. In such an embodiment, the composition may comprise from about 0.2 g to about 4 g of amino acid per from about 1 mg to about 20 mg of highly concentrated tocotrienols or tocotrienol derivatives. In one embodiment, the composition is provided in a tablet form or a powder plus aqueous solution form for oral consumption, and the composition is provided for administration from about two to about four times per day.

In one embodiment, the composition comprises from about 50 mg to about 200 mg L-arginine acid, from about 50 mg to about 200 mg L-glutamic acid, from about 50 mg to about 200 mg L-aspartic acid, and from about 25 mg to about 75 mg tocotrienol. In a further embodiment, the composition comprises about 100 mg L-aspartic acid, about 100 mg L-glutamic acid, about 100 mg L-arginine acid, and about 50 mg tocotrienol.

In one embodiment, the composition is dissolved in water. In a further embodiment, the composition is dissolved in a physiological saline solution. In a further embodiment, the composition is dissolved in a 5% sugar solution. In a further embodiment, the composition is dissolved in any physiologically safe neutral solvent.

In one embodiment, the composition comprises L-glutamic acid, L-aspartic acid, L-arginine acid, and tocotrienol, and the tocotrienol comprises about 20% to about 35% by weight of the total composition.

In one embodiment, the composition comprises a plurality of amino acids in equimolar proportions. In a further embodiment, the composition comprises a plurality of amino acids that are present in any proportion necessary to ensure that the final composition maintains a neutral pH.

### EXAMPLE 1

Chart 1 below shows amounts used for the treatment of organ failure. Components were dissolved in a physiological salt solution (transfusion) at a volume of 500 ml and administered via intravenous drip. The chart indicates highly concentrated tocotrienol derivatives were used in combination with three different amino acids (arginine, glutamine, aspartic acid; each in a sterile, 5 ml glass vial).

The following injection was used with patients suffering from any combination of brain, liver, and kidney failure as a result of poor circulation or heart failure. Patients were administered three doses per day, with each injection of 500 ml via intravenous drip taking one hour or longer.

CHART 1

| | Amount | Molarity | |
|---|---|---|---|
| Component | (grams/500 ml) | (mmol/500 ml) | Solubility |
| Highly concentrated | 0.001 | 0.0023 | 2 |
| tocotrienol derivatives | | | |
| Arginine | 1.74 | 10 | 15 |
| Glutamine | 1.47 | 10 | 2.6 |
| Aspartic acid | 1.33 | 10 | 0.45 |

The three amino acids were together added to meat in food and then given to the patient. Absorption neared 100%.

Of the three amino acids used, arginine was basic, glutamine was neutral, and aspartic acid was acidic. The three amino acids were combined in equimolar quantities to preserve a neutral pH in the final solution.

### EXAMPLE 2

Chart 2 indicates highly concentrated tocotrienol and the three listed amino acids combined in powdered form. The quantities in the chart indicate the dosage for one day, and that dose was divided into two equal doses per day.

CHART 2

| Component | Amount (mg) | Molarity (mmol) |
|---|---|---|
| Tocotrienol | 150 | 0.17 |
| Arginine | 1740 | 10 |
| Glutamine | 1470 | 10 |
| Aspartic acid | 1330 | 10 |

### EXAMPLE 3

Chart 3 indicates the diphenylpicrylhydrazl (DPPH) radical removal ability of torolox, tocotrienol, α-tocopherol, and coenzyme Q10. The reaction composition was carried out in a DPPH solution comprising 8 mg DPPH per 100 ml of 50% ethanol. The total reaction composition comprised 0.2 ml of the specimen (torolox, tocotrienol, α-tocopherol, or coenzyme Q10) and 1.8 ml of the aforementioned DPPH solution. The total reaction composition reacted at room temperature in a dark place for 30 minutes. The absorbance measurement was carried out at 517 nm. The results indicate that tocotrienol exhibited an inhibition rate at approximately the same level as a-tocopherol by this measurement, and that coenzyme Q10 did not exhibit inhibition action.

CHART 3

| | Absorbance | Inhibition | IC50 |
|---|---|---|---|
| Component | (517 | rate (%) | (μM) |
| Blank | 1.78 | | |
| Torolox 25 μM | 1.44 | 19.1 | 43.777 |
| Torolox 50 μM | 0.92 | 48.2 | |
| Torolox 100 μM | 0.09 | 94.9 | |
| Tocotrienol 25 μM | 1.49 | 16.3 | 55.022 |
| Tocotrienol 50 μM | 1.05 | 41 | |
| Tocotrienol 100 μM | 0.36 | 79.8 | |
| α-Tocopherol 25 μM | 1.51 | 15.2 | 51.06 |
| α-Tocopherol 50 μM | 1.05 | 41 | |
| | 0.22 | 87.6 | |
| Coenzyme Q10 | 2.02 | <0 | |
| | 2.6 | <0 | |
| | 2.13 | <0 | |

### EXAMPLE 4

Chart 4 indicates the linoleate peroxidative reaction inhibition action of tocotrienol, a-tocopherol, and Coenzyme Q10. The linoleate micellar solution comprised 300mM linoleate, 5 mM 2,2′-Azobis(2-amidinopropane) dihydrochloride (AAPH), and a specimen (tocotrienol, α-tocopherol, or Coenzyme Q10) dissolved in 0.3% Lubrol Px—20 mM sodium phosphate buffer (pH 7.4). The reaction composition reacted at room temperature for one hour, and then 0.5 ml of methanol was added to the composition, and then 2.0 ml of 70% methanol was added to the composition. The absorbance measurement was conducted at 234 nm. The results indicated that tocotrienol exhibited inhibition at the same level as a-tocopherol, and that Coenzyme Q10 did not exhibit inhibition action.

CHART 4

| | Absorbance (234 nm) | Inhibition rate (%) |
|---|---|---|
| Control | 2.18 | |
| Tocotrienol 25 μM | 1.26 | 42.2 |
| α-Tocopherol 25 μM | 1.26 | 42.2 |
| Coenzyme Q10 25 μM | 2.17 | — |

Example 5 is a composition for improving blood circulation in a user. The composition comprises an effective amount of tocotrienol and an effective amount of at least one of an amino acid selected from a group comprising arginine, aspartic acid, glutamine, glutamic acid, proline, serine, cysteine, histidine, tryptophan, and tyrosine.

Example 6 is a composition as in example 5, wherein the composition is in a tablet or powder form.

Example 7 is a composition as in example 5, wherein the composition is prepared for intravenous administration or intramuscular administration. The composition may be dissolved in a neutral solvent.

Example 8 is a composition as in example 5, wherein the composition comprises a plurality of the amino acids selected from the group comprising arginine, aspartic acid, glutamine, glutamic acid, proline, serine, cysteine, histidine, tryptophan, and tyrosine, and wherein the plurality of amino acids are present in the proportions necessary for an overall neutral composition.

Example 9 is a composition as in example 5, wherein the amino acids comprise aspartic acid, arginine, and glutamic acid.

Example 10 is a composition as in example 5, wherein the tocotrienol comprises from about 20% to about 35% by weight of the total composition.

Example 11 is a composition as in example 5, wherein the composition is dissolved in a solvent, and wherein the composition comprises from about 0.2 g to about 4 g of amino acid per 100 mL of solvent.

Example 12 is a composition as in example 5, wherein the composition is dissolved in a solvent, and wherein the composition comprises from about 0.5 mg to about 2 mg tocotrienol per from about 200 mL to about 500 mL of solvent.

Example 13 is a method of improving blood circulation in a user. The method comprises providing a composition to the user comprising an effective amount of tocotrienol and an effective amount of at least one of an amino acid selected from a group comprising arginine, aspartic acid, glutamine, glutamic acid, proline, serine, cysteine, histidine, tryptophan, and tyrosine.

Example 14 is a method as in example 13, wherein the composition is provided for intravenous administration or intramuscular administration. Further, the composition may be provided for acute organ failure.

Example 15 is a method as in example 13, wherein the composition is provided in a tablet or powder form. Further, the composition may be provided for the treatment of a chronic condition. The chronic condition may concern blood circulation or cellular respiration. The chronic condition may comprise, for example, chronic tissue hypoxemia, organ damage as a result of cell death, dementia, ulceration of the skin, chronic failure of the liver, kidneys, heart, brain, skin, and other organs. The composition may be provided for preventing the progression of further tissue damage by increasing blood circulation.

Example 16 is a method as in example 13, wherein the amino acids comprise aspartic acid, arginine, and glutamic acid.

Example 17 is a method as in example 13, wherein the tocotrienol comprises from about 20% to about 35% by weight of the total composition.

Example 18 is a method as in example 13, wherein the composition is dissolved in either of a physiological saline solution or a 5% sugar solution.

Example 19 is a method as in example 13, wherein the composition is prepared for intravenous or intramuscular administration, and wherein the composition comprises from about 0.2 g to about 4 g of amino acid per 100 mL of solution.

Example 20 is a method as in example 13, wherein the composition is prepared for intravenous or intramuscular administration, and wherein the composition comprises from about 0.5 mg to about 2 mg of tocotrienol per about 200 mL to about 500 mL of solution.

Example 21 is a composition for improving blood circulation in a user. The composition comprises tocotrienol, L-aspartic acid, L-arginine acid, and L-glutamic acid, and wherein the tocotrienol comprises from bout 20% to about 35% by weight of the total composition.

Example 21 is a composition for improving cell life in a hypoxic condition. The composition comprises an effective amount of tocotrienol and an effective amount of at least one of an amino acid selected from a group comprising arginine, aspartic acid, glutamine, glutamic acid, proline, serine, cysteine, histidine, tryptophan, and tyrosine.

According to one or more embodiments of the disclosure, a composition may include a combination of all or some, but not all, of the following ingredients:

(a) tocotrienol;

(b) arginine;

(c) aspartic acid;

(d) glutamine;

(e) glutamic acid;

(f) proline;

(g) serine;

(h) cysteine;

(i) histidine;

(j) tryptophan; and

(k) tyrosine.

Other embodiments of the composition may comprise, for example, concentrations of tocotrienol as follows:

(a1) from 20% to 35% by weight the total composition;

(a2) from 21% to 34% by weight the total composition;

(a3) from 22% to 33% by weight the total composition;

(a4) from 23% to 32% by weight the total composition;

(a5) from 24% to 31% by weight the total composition;

(a6) from 25% to 30% by weight the total composition;

(a7) from 26% to 29% by weight the total composition;

(a8) from 26% to 28% by weight the total composition;

(a9) from 25% to 29% by weight the total composition;

(a10) from 25% to 28% by weight the total composition.

With respect to ingredient (b) noted above for example, the amount of arginine that may be included in the final composition is based on a percent by weight of the total weight of the final composition described herein. The composition may comprise ingredient (b) for example, in concentrations as follows:

(b1) from 15% to 55% by weight the total composition;

(b2) from 16% to 54% by weight the total composition;

(b3) from 17% to 53% by weight the total composition;

(b4) from 18% to 52% by weight the total composition;

(b5) from 19% to 51% by weight the total composition;

(b6) from 20% to 50% by weight the total composition;

(b7) from 21% to 49% by weight the total composition;

(b8) from 22% to 48% by weight the total composition;

(b9) from 23% to 47% by weight the total composition;

(b10) from 23% to 46% by weight the total composition;

(b11) from 23% to 45% by weight the total composition;

(b12) from 23% to 44% by weight the total composition;

(b13) from 23% to 43% by weight the total composition;

(b14) from 23% to 42% by weight the total composition;

(b15) from 23% to 41% by weight the total composition;

(b16) from 23% to 40% by weight the total composition;

(b17) from 23% to 39% by weight the total composition;

(b18) from 23% to 38% by weight the total composition;

(b19) from 23% to 37% by weight the total composition;

(b20) from 23% to 36% by weight the total composition;

(b21) from 23% to 35% by weight the total composition;

(b22) from 23% to 34% by weight the total composition;

(b23) from 23% to 33% by weight the total composition.

With respect to ingredient (c) noted above for example, the amount of aspartic acid that may be included in the final composition is based on a percent by weight of the total weight of the final composition described herein. The composition may comprise ingredient (c) for example, in concentrations as follows:

(c1) from 15% to 55% by weight the total composition;

(c2) from 16% to 54% by weight the total composition;

(c3) from 17% to 53% by weight the total composition;

(c4) from 18% to 52% by weight the total composition;

(c5) from 19% to 51% by weight the total composition;

(c6) from 20% to 50% by weight the total composition;

(c7) from 21% to 49% by weight the total composition;

(c8) from 22% to 48% by weight the total composition;

(c9) from 23% to 47% by weight the total composition;

(c10) from 23% to 46% by weight the total composition;

(c11) from 23% to 45% by weight the total composition;

(c12) from 23% to 44% by weight the total composition;

(c13) from 23% to 43% by weight the total composition;

(c14) from 23% to 42% by weight the total composition;

(c15) from 23% to 41% by weight the total composition;

(c16) from 23% to 40% by weight the total composition;

(c17) from 23% to 39% by weight the total composition;

(c18) from 23% to 38% by weight the total composition;

(c19) from 23% to 37% by weight the total composition;

(c20) from 23% to 36% by weight the total composition;

(c21) from 23% to 35% by weight the total composition;

(c22) from 23% to 34% by weight the total composition;

(c23) from 23% to 33% by weight the total composition.

With respect to ingredient (e) noted above for example, the amount of glutamic acid that may be included in the final composition is based on a percent by weight of the total weight of the final composition described herein. The composition may comprise ingredient (e) for example, in concentrations as follows:

(e1) from 15% to 55% by weight the total composition;

(e2) from 16% to 54% by weight the total composition;

(e3) from 17% to 53% by weight the total composition;

(e4) from 18% to 52% by weight the total composition;

(e5) from 19% to 51% by weight the total composition;

(e6) from 20% to 50% by weight the total composition;

(e7) from 21% to 49% by weight the total composition;

(e8) from 22% to 48% by weight the total composition;

(e9) from 23% to 47% by weight the total composition;

(e10) from 23% to 46% by weight the total composition;

(e11) from 23% to 45% by weight the total composition;

(e12) from 23% to 44% by weight the total composition;

(e13) from 23% to 43% by weight the total composition;

(e14) from 23% to 42% by weight the total composition;

(e15) from 23% to 41% by weight the total composition;

(e16) from 23% to 40% by weight the total composition;

(e17) from 23% to 39% by weight the total composition;

(e18) from 23% to 38% by weight the total composition;

(e19) from 23% to 37% by weight the total composition;

(e20) from 23% to 36% by weight the total composition;

(e21) from 23% to 35% by weight the total composition;

(e22) from 23% to 34% by weight the total composition;

(e23) from 23% to 33% by weight the total composition.

Also, according to one or more non-limiting embodiments of the disclosure, any of the concentrations for ingredients (b), (c), or (e), for example, as listed above, may indicate the concentration for any of ingredients (b) thru (k), as listed above. For example, an embodiment of the disclosure may comprise, for example, (a6) from 25% to 30% by weigh the total composition of tocotrienol, and equal parts by molarity of arginine, aspartic acid, and glutamic acid. For example, an embodiment of the disclosure may comprise, for example (a6) from 25% to 30% by weight the total composition of tocotrienol, from 23% to 33% by weight the total composition glutamic acid, from 23% to 33% by weight the total composition tryptophan, and from 23% to 33% by weight the total composition histidine. For example, the composition may comprise all, or any combination of but not all, of the ingredients (a) thru (k). For example, the composition may comprise concentrations of any of the amino acids (b) thru (k) in any of the concentrations listed for (b) arginine, (c) aspartic acid, or (e) glutamic acid. For example, the composition may comprise tocotrienol and each of the amino acids (b) thru (k).

In one embodiment, a method of improving blood circulation in a user is provided. The method comprises: providing a composition to the user comprising an effective amount of tocotrienol and an effective amount of at least one of an amino acid selected from a group comprising arginine, aspartic acid, glutamine, glutamic acid, proline, serine, cysteine, histidine, tryptophan, and tyrosine. In one embodiment, the composition is provided for intravenous administration. In a further embodiment, the composition is provided for intramuscular administration. In a further embodiment, the composition is provided in a tablet form or a powdered form.

In one embodiment, the composition is provided for the treatment of acute organ failure. In such an embodiment, the composition may be administered intravenously. In one embodiment, the composition is provided for the treatment of a chronic condition concerning blood circulation or cellular respiration. In such an embodiment, the composition may be administered on a regular basis in a tablet form or a powdered form. The composition may be administered, for example, every day, multiple times per day, every week, every month, or in any other suitable interval.

The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations might be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A composition for improving blood circulation in a user, comprising:
an effective amount of tocotrienol; and
an effective amount of at least one of an amino acid selected from a group comprising arginine, aspartic acid, glutamine, glutamic acid, proline, serine, cysteine, histidine, tryptophan, and tyrosine.

2. The composition of claim 1, wherein the composition is in a tablet or powder form.

3. The composition of claim 1, wherein the composition is prepared for intravenous administration.

4. The composition of claim 3, further comprising a neutral solvent.

5. The composition of claim 1, wherein the composition comprises a plurality of the amino acids selected from the group comprising arginine, aspartic acid, glutamine, glutamic acid, proline, serine, cysteine, histidine, tryptophan, and tyrosine, and wherein the plurality of amino acids are present in proportions necessary for the composition to have a neutral pH.

6. The composition of claim 1, wherein the amino acids comprise aspartic acid, arginine, and glutamic acid.

7. The composition of claim 1, wherein the tocotrienol comprises from about 20% to about 35% by weight of the total composition.

8. The composition of claim 1, wherein the composition is dissolved in a solvent, and wherein the composition comprises from about 0.2 g to about 4 g of amino acid per 100 mL of the solvent.

9. The composition of claim 1, wherein the composition is dissolved in a solvent, and wherein the composition comprises from about 0.5 mg to about 2 mg tocotrienol per from about 200 mL to about 500 mL of the solvent.

10. A method of improving blood circulation in a user, comprising:
providing a composition to the user comprising an effective amount of tocotrienol and an effective amount of at least one of an amino acid selected from a group consisting essentially of arginine, aspartic acid, glutamine, glutamic acid, proline, serine, cysteine, histidine, tryptophan, and tyrosine.

11. The method of claim 10, wherein the composition is provided for intravenous administration or intramuscular administration.

12. The method of claim 11, wherein the composition is provided for the treatment of acute organ failure.

13. The method of claim 10, wherein the composition is provided in a tablet or powder form.

14. The method of claim 13, wherein the composition is provided for the treatment of a chronic condition.

15. The method of claim 10, wherein the amino acids comprise aspartic acid, arginine, and glutamic acid.

16. The method of claim 10, wherein the tocotrienol comprises from about 20% to about 35% by weight of the total composition.

17. The method of claim 10, wherein the composition is dissolved in either of a physiological saline solution or a 5% sugar solution.

18. The method of claim 10, wherein the composition is prepared for intravenous or intramuscular administration, and wherein the composition comprises from about 0.2 g to about 4 g of amino acid per 100 mL of solution.

19. The method of claim 10, wherein the composition is prepared for intravenous or intramuscular administration, and wherein the composition comprises from about 0.5 mg to about 2 mg of tocotrienol per about 200 mL to about 500 mL of solution.

20. A composition for improving blood circulation in a user, comprising:
tocotrienol;
L-aspartic acid;
L-arginine acid; and
L-glutamic acid; and
wherein the tocotrienol comprises from about 20% to about 35% by weight of the total composition.
